(19) United States
(12) Patent Application Publication
(10) Pub. No.: US 2008/0254056 A1
(43) Pub. Date: Oct. 16, 2008

(54) Green Alga Extract with High Astaxanthin Content and Method of Producing the Same

(75) Inventor: Kai Zhang, Shizuoka (JP)

Correspondence Address: McLELAND PATENT LAW OFFICE, P.L.L.C., 11320 RANDOM HILLS ROAD, SUITE 250, FAIRFAX, VA, 22030, US

(73) Assignee: YAMAHA HATSUDOKI KABUSHIKI KAISHA, Iwata-shi, Shizuoka (JP)

(21) Appl. No.: 11/720,116

(22) PCT Filed: Aug. 29, 2006
(86) PCT No.: PCT/JP2006/317414

§ 371 Date: Aug. 1, 2007

(30) Foreign Application Priority Data
Sep. 6, 2005 (JP) 2005-258300

(51) Int. Cl. C12P 23/00; A61K 36/05

(52) U.S. Cl. 424/195.17; 435/67

(57) ABSTRACT

A green algal extract that contains astaxanthin at a concentration of 8 wt % or more can be obtained by cultivating an encysted green alga in a nutrient medium while supplying carbon dioxide and providing irradiation with light at a photosynthetically active photon flux input of 8000 μmol-photon/m³/s or more, and extracting an oil component, which contains astaxanthin. A green alga that belongs to the genus Haematococcus is preferable.

### TECHNICAL FIELD

The present invention relates to efficient production of astaxanthin. More specifically, the present invention relates to green algal extracts with a high astaxanthin content and methods for producing the same.

### BACKGROUND ART

Astaxanthin is a carotenoid imparting a red color, and is known to have potent antioxidative effect. For this reason, it is used as a pigment in food, a cosmetic, a health food product, and a pharmaceutical. Some astaxanthins are chemically synthesized, and astaxanthins are also naturally occurring. Naturally occurring astaxanthins are extracted, for example, from Eucarida such as euphausiids and Pandalus borealis, from Phaffia yeast, and from algae. However, astaxanthin cannot be produced efficiently from Eucarida such as euphausiids or from yeast because of their low astaxanthin content.

On the other hand, algae are encysted as a result of a change in the external environment and accumulate astaxanthin within algal cells. Thus, production of astaxanthin from algae has been investigated. Fabregas et al. in J. Biotech., Vol. 89, p. 65 (2001) describe a two-step culture in which vegetative cells of Haematococcus are obtained while exchanging 10 to 40% of a culture medium every day (i.e., fed-batch culture) and then batch culture is performed for an additional 15 days under irradiation with light. Japanese Laid-Open Patent Publication (Tokuhyo) 2-501189 describes a process of producing astaxanthin by cultivating Haematococcus by varying the ratio of carbon and nitrogen, which are components of a culture medium, at a late stage of the cultivation. Moreover, Japanese Laid-Open Patent Publication No. 1-187082 describes a method for producing astaxanthin by cultivating algae in a culture medium supplemented with a metal salt. However, these documents do not describe the content of astaxanthin in the algal cells.

Japanese Laid-Open Patent Publication No. 3-83577 discloses an astaxanthin content of 0.3 to 10 wt % in dry algal cells, but in an example in which algal cells were cultivated under limited nitrogen supply and under culture conditions of 40000 lux, the astaxanthin content was about 2 wt %, and algal cells having an astaxanthin concentration of 10 wt % or more were not actually obtained. Furthermore, Japanese Laid-Open Patent Publication No. 2000-60532 discloses that Haematococcus cultivated in an outdoor culture pool contained 4.5 wt % astaxanthin in algal cells.

The astaxanthin content per algal cell also has been studied, and for example, Japanese Laid-Open Patent Publication No. 7-39389 and Tjahjono et al., BIOTECHNOLOGY LETTERS, Vol. 16, pp. 133-138 (1994) disclose that astaxanthin was produced at about 600 pg/cell through cultivation in the presence of iron ions and acetic acid at a culture temperature of 30° C. Further, Japanese Laid-Open Patent Publication No. 2004-129504 discloses that it is possible to increase the amount of astaxanthin to a value of about 700 pg/cell or more. However, in practice, algae containing astaxanthin at such a high concentration have not been obtained, and even the highest value in the examples described in Japanese Laid-Open Patent Publication No. 2004-129504 was only 156 pg/cell.

WO 2005/116238 discloses a method for efficiently producing xanthophyll by inoculating microalgae containing xanthophyll, e.g., encysted microalgae, into a nutrient medium to grow the microalgae as vegetative cells and further encysting the grown microalgae. In the examples in WO 2005/116238, the highest xanthophyll (astaxanthin) content in the dry algal cells was 3.5 wt %.

In order to efficiently produce astaxanthin, there has been a demand for algae containing astaxanthin at a higher concentration.

### DISCLOSURE OF THE INVENTION

The present invention provides a green algal extract, which contains astaxanthin at a concentration of 8 wt % or more.

The present invention also provides a method for producing a green algal extract that contains astaxanthin at a concentration of 8 wt % or more, the method comprising:

cultivating an encysted green alga in a nutrient medium while supplying carbon dioxide and providing irradiation with light at a photosynthetically active photon flux input of 8000 μmol-photon/m³/s or more; and

extracting an oil component that contains astaxanthin.

The present invention further provides a method for producing astaxanthin, the method comprising:

cultivating an encysted green alga in a nutrient medium while supplying carbon dioxide and providing irradiation with light at a photosynthetically active photon flux input of 8000 μmol-photon/m³/s or more;

extracting an oil component that contains astaxanthin; and

recovering astaxanthin from the extracted oil component that contains astaxanthin.

In a particular embodiment, the nutrient medium is an autotrophic medium.

In one embodiment, the photosynthetically active photon flux input is not more than 240000 μmol-photon/m³/s.

In an embodiment, the green alga is a unicellular alga belonging to the genus Haematococcus.

In another embodiment, the green alga is Haematococcus pluvialis.

In one embodiment, the green alga is cultivated in an autotrophic medium.

The present invention also provides a green algal extract obtained from a dried green alga that contains astaxanthin at a concentration of 3.1 wt % or more. The green algal extract contains astaxanthin at a concentration of 8 wt % or more.

According to the present invention, a green algal extract that contains astaxanthin at a concentration of 8 wt % or more is provided. Since the astaxanthin concentration of the extract is high, the extract can be used as is as an ingredient for food or medicine without further purification or concentration thereof. Further, the production efficiency of purified astaxanthin can be improved more than ever before by using the green algal extract of the present invention.

### BRIEF DESCRIPTION OF DRAWINGS

FIG. 1 is a schematic vertical cross section of the culture bath where cultivation is performed, in one example of the culture apparatus used in the present invention.

FIG. 2 is a graph showing the change in the astaxanthin concentration of the green algal extract over time.

### BEST MODE FOR CARRYING OUT THE INVENTION

### Green Algae

There is no particular limitation on the green algae used in the present invention, as long as the green algae can produce astaxanthin. For example, unicellular algae belonging to the genus Haematococcus are preferably used. Examples of the green algae include Haematococcus pluvialis (H. pluvialis), Haematococcus lacustris (H. lacustris), Haematococcus capensis (H. capensis), Haematococcus droebakensi (H droebakensi), and Haematococcus zimbabwiensis (H zimbabwiensis).

Examples of Haematococcus pluvialis (H. pluvialis) include the NIES144 strain deposited in the Independent Administrative Institution National Institute for Environmental Studies, the UTEX2505 strain deposited in the Culture Collection of Algae at the University of Texas, U.S.A., and the K0084 strain deposited in the Scandinavian Culture Center for Algae and Protozoa, Botanical Institute, at the University of Copenhagen, Denmark.

Examples of Haematococcus lacustris (H lacustris) include the ATCC30402 and ATCC30453 strains deposited in ATCC, the IAM C-392, IAM C-393, IAM C-394, and IAM C-339 strains deposited in the Institute of Molecular and Cellular Biosciences, University of Tokyo, or the UTEX16 and UTEX294 strains.

Examples of Haematococcus capensis (H. capensis) include the UTEX LB1023 strain.

Examples of Haematococcus droebakensi (H. droebakensi) include the UTEX55 strain.

Examples of Haematococcus zimbabwiensis (H. zimbabwiensis) include the UTEX LB1758 strain.

Among these, Haematococcus pluvialis is preferably used.

### (Encystment)

In the present invention, the green algae described above that contain astaxanthin are used. When the green algae are subjected to stresses from the environment, such as nutrient deprivation or the presence of oxides, the green algae accumulate astaxanthin within the cells and become resting spores. The shift to this resting state is referred to as encystment. In this specification, encystment refers to any state from the beginning of the resting state where accumulation of astaxanthin starts, to the completely encysted state where the cells become resting spores. In order to increase the astaxanthin content, it is preferable to use green algae in which encystment has progressed as far as possible and which has accumulated a large amount of astaxanthin. It should be noted that “cultivating encysted green algae” as used herein also includes the process of inoculating green algae containing astaxanthin that has been grown in a nutrient medium, after the green algae has reached the encysted state. In the present specification, “green algae” are also intended to include encysted green algae.

### (Medium)

There is no particular limitation on the medium used to cultivate the green algae. Generally, a medium is used that contains nitrogen, inorganic salts of trace metal (e.g., phosphorous, potassium, magnesium, and iron), vitamins (e.g., thiamine), and the like, which are essential to growth. For example, media such as the VT medium, C medium, MC medium, MBM medium, and MDM medium (see Sorui Kenkyuho, ed. by Mitsuo Chihara and Kazutoshi Nishizawa, Kyoritsu Shuppan (1979)), the OHM medium (see Fabregas et al., J. Biotech., Vol. 89, pp. 65 (2001)), the BG-11 medium, and modifications thereof may be used. In the present invention, it is preferable to use an autotrophic medium that is substantially free from organic carbon source so that contamination by bacteria can be prevented.

These media may be selected depending on their purposes, such as growth, or encystment. For example, for growth of the green algae, a medium having a large amount of components serving as a nitrogen source is used (rich medium: containing at least 0.15 g/L expressed in terms of nitrogen). For encystment, a medium having a small amount of components serving as a nitrogen source is used (encystment medium: containing less than 0.02 g/L expressed in terms of nitrogen). Alternatively, a medium containing a nitrogen source at an intermediate concentration between these media may be used (low nutrient medium: containing at least 0.02 g/L and less than 0.15 g/L expressed in terms of nitrogen).

The nitrogen source concentration, phosphorous concentration, and other properties of the medium can be determined depending on the amount of the green algae to be inoculated. For example, when a green algae count in the order of 10⁵ is inoculated in a low nutrient medium, the green algae would grow to a certain extent, but the growth may stop soon because the amount of the nitrogen source is too small. Such a low nutrient medium is suitable for performing growth and encystment continuously in a single step (in a batch manner), as described later. Furthermore, by adjusting the N/P mole ratio to value from 10 through 30, preferably 15 through 25, the green alga can be encysted.

In the case where the green algae count for inoculation is even higher, the rich medium can be employed to perform the above-described cultivation.

In this manner, the composition of the medium can be determined in consideration of various conditions. It should be noted that the medium preferably used in the present invention, i.e., an autotrophic medium, is nearly free from an organic carbon source such as acetic acid or glucose, so that contamination by bacteria hardly occurs even in long-term cultivation.

### (Culture Apparatus)

There is no particular limitation on the apparatus for cultivating the green algae, as long as the apparatus is capable of supplying carbon dioxide and irradiating a culture suspension with light. For example, in the case of a small-scale culture, a flat culture flask may be preferably used. In the case of a large-scale culture, a culture tank that is constituted by a transparent plate made of glass, plastic, or the like and that is equipped with an irradiation apparatus and an agitator, if necessary, may be used. Examples of such a culture tank include a plate culture tank, a tube-type culture tank, an airdome-type culture tank, and a hollow cylinder-type culture tank. In any case, a sealed container is preferably used.

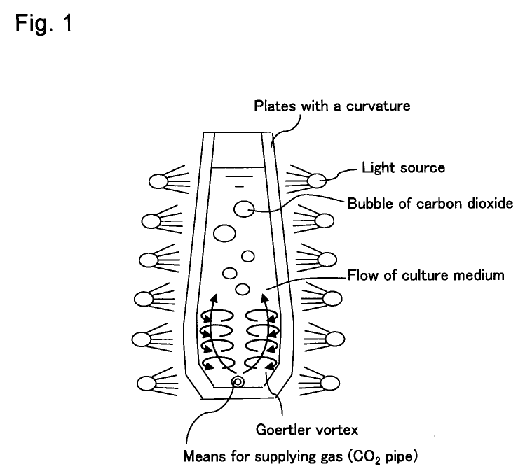

In the present invention, as a culture apparatus, for example, it is preferable to use a flat culture apparatus provided with a housing made of a pair of plates in opposition to one another with a predetermined distance between them, and each of the pair of plates has a curvature that bulges outward (see FIG. 1). Due to the presence of the curved portions in such a flat culture apparatus, it is possible to develop a Goertler vortex in the culture medium. Because the Goertler vortex occurs substantially perpendicular to the vertical flow of the culture medium (that is, the vortex occurs horizontally), the culture medium moves up and down vertically while being swirled horizontally. Thus, not only is the agitation efficiency increased, but it is also possible to prevent the green algae from adhering to the wall surface.

### (Culture Conditions)

There is no particular limitation on the culture conditions, and a temperature, a pH, and the like as generally employed for cultivation of green algae can be used. The green algae are cultivated at, for example, 15 to 35° C., and preferably 20 to 25° C. It is preferable that the pH is maintained at 6 to 8 throughout the cultivation period. Carbon dioxide is supplied by bubbling a gas containing carbon dioxide at a concentration of 1 to 3 vol % at a rate of 0.2 to 2 vvm, for example. When a plate culture tank is used, the culture suspension is stirred by supplying carbon dioxide, so that the green algae can be uniformly irradiated with light.

### (Light Irradiation)

In the present invention, the green algae are cultivated by irradiating the green algae with light so that the photosynthetically active photon flux input is 8000 μmol-photon/m³/s (hereinafter, this unit is abbreviated as μmol-p/m³/s) or more, preferably 12000 μmol-p/m³/s or more, more preferably 25000 μmol-p/m³/s or more. Moreover, since cell growth is stopped when photosynthesis is inhibited by intense light, the photosynthetically active photon flux input is preferably not more than 240000 μmol-p/m³/s. The amount of astaxanthin produced is significantly increased by performing irradiation with light at such a photosynthetically active photon flux input throughout the entire cultivation process from the start of cultivation to encystment.

The photosynthetically active photon flux input can be obtained by first measuring the photosynthetically active photon flux density (PPFD). The PPFD can be obtained by placing a flat-surface photon sensor LI-190 (LICOR Inc., Lincoln, USA) at several points in the culture apparatus, performing irradiation with light to measure the PPFD at each of the points, and averaging the values obtained. When there is a plurality of light sources, the PPFD is the total of each PPFD received from the respective light sources. When the apparatus is constituted by a transparent plate such as glass or an acrylic resin, a light source may be positioned by measuring the PPFD passing through the transparent plate, and then determining the intensity of the light source or the distance of the light source required for obtaining a predetermined PPFD. When a culture tank constituted by two transparent flat plates is used and irradiation with light is performed from both sides of the culture tank, the PPFD is obtained by adding up the PPFD values from the respective sides.

The photosynthetically active photon flux input can be calculated using the following equation:

$$Photosynthetically active photon flux input = \frac{\begin{matrix}PPFD ( μ mol / {m}^{2} / s ) \times \\ Light receiving area ( {m}^{2} )\end{matrix}}{Volume of alga culture ( {m}^{3} )}$$

### (Culture Method)

Cultivation is performed under irradiation with light by selecting appropriately and combining the above-described medium, culture apparatus, culture conditions, and the like. There are two culture methods. One method is a single-step culture in which encysted green algae are grown and encysted continuously in the same medium. The other method is a two-step culture in which the medium for growing encysted green algae and the medium for encysting the grown green algae are different from each other, and growth and encystment are performed separately.

### (Single-Step Culture)

The single-step culture is a method of cultivating the encysted green algae continuously without changing the medium during the period from inoculation of the encysted green algae into a nutrient medium to the end of the cultivation. In other words, growth and encystment of the green algae are performed with a predetermined medium in the same culture tank. In this single-step culture, once the green algae have grown, the green algae are shifted to an encysted state smoothly under any of the following stresses: nutrient starvation stress due to consumption of the nutrients in the medium, stress due to irradiation with light. The encysted green algae obtained by this single-step culture may be further used for a subsequent single-step culture or for inoculation into a medium for use in cultivation of the two-step culture described later.

When a green alga containing astaxanthin, preferably an encysted green alga, is inoculated into the nutrient medium, the green alga releases 2ⁿ (n=1 to 4) zoospores containing astaxanthin. These zoospores become vegetative cells which still contain astaxanthin, so that the number of vegetative cells containing astaxanthin increases (i.e., the green algae can grow). By encysting vegetative cells containing astaxanthin, astaxanthin is newly accumulated in addition to the astaxanthin originally contained in the green algae, and thus the astaxanthin content in the cells is increased even more.

It is believed that when the vegetative cells continue to grow, the astaxanthin concentration in the vegetative cells eventually decreases, and therefore, it is preferable to stop growth at a point where some astaxanthin remains in the cells.

In order to stop growth of the green alga at the point where the vegetative cells have grown to a given extent, it is preferable that the medium be designed to cause nutrient-starvation. For this purpose, in the single-step culture, a medium having a relatively low nitrogen source concentration, e.g., the low nutrient medium described above, is preferably used. When a large amount of encysted cells is inoculated, a medium having a high nitrogen source concentration, e.g., the rich medium described above, may be used.

It should be noted that when the green algae do not grow sufficiently in the low nutrient medium, a rich medium or a low nutrient medium may be supplemented so as to grow the green algae to a given extent.

Moreover, in the case where a low nutrient medium is used, if the N/P mole ratio is adjusted to a value between 10 and 30, preferably between 15 and 25, then the green algae can be encysted smoothly after growth.

The single-step culture not only has advantages such as that process control can be performed easily, contamination by bacteria can be prevented because transfer to another culture tank is not necessary, and the method can be performed with a single culture tank, but also that it is possible to obtain readily green algae containing astaxanthin at a high concentration.

### (Two-Step Culture)

The two-step culture is a culture method in which encysted green algae are grown in a nutrient medium, and then encysted by changing the nutrient medium to an encystment medium. In other words, the two-step culture includes a first step in which encysted green algae are first inoculated into a nutrient medium, preferably a rich medium, to grow the green algae, and a second step in which the green algae are collected and transferred to an encystment medium that is nearly free from a nitrogen source, and then encysted.

Since it is necessary to finish growth of the green algae in the first step while astaxanthin remains in the vegetative cells, cultivation in the nutrient medium is performed for a short period of time. When the cultivation is performed using a rich medium at the start of the cultivation, the growth rate of the vegetative cells is higher than the growth rate when a low nutrient medium is used, and therefore it is preferable to use a rich medium. After growth, the green algae are collected and transferred to an encystment medium, and encysted in the second step.

The first step and the second step may be performed independently in a batch manner using separate culture tanks. It is also possible to wash and collect the grown green algae at the end of the first step, place the green algae back in the same culture tank, and then perform the second step.

With this two-step culture, green algae having high astaxanthin content can also be obtained. This two-step culture has an advantage in that the growth step can be finished in a shorter period of time than with the single-step culture; however, the operation of transferring the grown green algae is required in the two-step process.

It is also possible to use a portion of the obtained encysted green algae for collecting the green algal extract containing astaxanthin and a portion of the remainder for another inoculation into a nutrient medium.

By the above-described cultivation, a green algae culture suspension containing astaxanthin (expressed in terms of free form) at a concentration of 150 mg or more per 1 L of the culture suspension, preferably 150 to 250 mg/L, and more preferably 200 to 250 mg/L can be obtained. The amount of astaxanthin per culture suspension can be determined by collecting a predetermined volume of the culture suspension and measuring the amount of astaxanthin contained in the collected culture suspension.

(Green Algal Extract Containing Astaxanthin at a Concentration of 8 wt % or more)

The green algal extract of the present invention contains astaxanthin (expressed in terms of its free form) at a concentration of 8 wt % or more, preferably 9 wt % or more, more preferably 10 wt % or more, and even more preferably between 12 to 14 wt %, or greater than 10 wt % but not more than 14 wt %.

In the present invention, a “green algal extract” refers to the oil component contained in the green algae described above, and refers to the oil component in which the astaxanthin has not been subjected to concentration or purification outside of the extraction operation.

The method for producing the green algal extract includes a step of culturing encysted green algae in a nutrient medium with a supply of carbon dioxide and under irradiation with light at a photosynthetically active photon flux input of 8000 μmol-p/m³/s or more, and a step of extracting the oil component, which contains astaxanthin. The cultivation step of the green algae is as described above.

There are no particular limitations regarding the means for extracting the oil component, which contains astaxanthin, and means ordinarily employed by those skilled in the art may be adopted. For example, first, green algae cultivated by the above-described method are dried by a drying means commonly used by those skilled in the art (e.g., drum drying, hot air drying, spray drying, or freeze-drying), so that a dry product of the green algae is obtained. Then, extraction by solvent, mechanical breaking (with a bead beater, for example), squeezing, or extraction by a combination thereof is performed. As the solvent, it is possible to use an organic solvent such as chloroform, hexane, acetone, methanol, or ethanol. Alternatively, the oil component may be extracted by supercritical extraction. When a solvent is used for the extraction, the solvent is removed after the extraction by means usually employed by those skilled in the art.

The dry product of green algae used in the extraction (that is, the dry algal cells) contains astaxanthin (expressed in terms of its free form) at a concentration of 3.1 wt % or more. Preferably it contains astaxanthin at a concentration of 5 wt % or more, more preferably 5 to 8 wt %, and even more preferably 6 to 7 wt %. It should be noted that the water content of the dry product of the green algae as used herein is 7 wt % or less, preferably 5 wt % or less, and more preferably about 2 wt %. When such a dry product of the green algae is used, it is possible to obtain a green algal extract that contains astaxanthin at a concentration of 8 wt % or more.

### Method for Producing Astaxanthin

The method of the present invention for producing astaxanthin further includes a step of recovering astaxanthin from the oil component that contains astaxanthin (that is, the green algal extract) obtained through the above-described method for producing the green algal extract. For example, astaxanthin may be produced by subjecting the green algal extract containing astaxanthin to separation and purification using means commonly used by those skilled in the art such as crystallization or fractionation using a synthetic resin (e.g., styrene-divinylbenzene copolymer) to recover and purify astaxanthin.

### EXAMPLES

Hereinafter, the present invention will be described through examples in which the Haematococcus pluvialis K0084 strain is used, but the present invention is not limited to these examples. It should be noted that in the examples, the amount of astaxanthin, the number of cells, and the amount of dry product of algal cells were measured according to the following methods.

### (Measurement of the Amount of Astaxanthin)

The amount of astaxanthin was measured by the following method. First, a given amount of the sample was collected, washed, and taken into a microtube designed for exclusive use with a bead beater. After adding zirconia beads to the tube, acetone was added thereto, and the sample was beaten with the bead beater. After the beating step, the sample was separated into a supernatant and a precipitate by centrifugation, and the supernatant (i.e., an acetone fraction) was collected. Acetone was added again to the precipitate, and the same operation as described above was repeated until the color of the precipitate became almost completely white. The collected acetone fractions were combined and diluted 100-fold with dimethyl sulfoxide (DMSO), and the absorbance at 492 nm (A₄₉₂) and the absorbance at 750 nm (A₇₅₀) were measured. The astaxanthin concentration in the collected acetone fractions (sample) was calculated using the following equation, and the amount of astaxanthin in the culture suspension was obtained from this calculated value. It should be noted that the amount of astaxanthin in the extract was obtained by diluting the extract with DMSO appropriately, then measuring the absorbance at 492 nm (A₄₉₂) and the absorbance at 750 nm (A₇₅₀) of the diluted extract, and calculating the astaxanthin concentration to obtain the astaxanthin concentration before dilution from this calculated value:

Astaxanthin concentration in sample(μg/mL)=4.5×100×(A₄₉₂−A₇₅₀)

### (Measurement of the Number of Cells)

The number of cells was measured using a particle size distribution measurement device (SYSMEX FPI-3000).

### (Measurement of the Dry Weight of Algal Cells)

The method for measuring the dry weight of green algae was as follows. First, a given volume of a culture suspension was collected and filtered on a GC50 glass fiber filter (made by Advantec Toyo Kaisha, Ltd.) under reduced pressure, and then washed twice with 5 mL of an aqueous solution of hydrochloric acid at pH 4 to dissolve inorganic salts. Then, the filter to which the green algae were adsorbed was dried in a thermostatic drier at 105° C. for 3 hours and cooled in a vacuum desiccator for one hour to room temperature, and then the weight of the dried filter was measured. The weight of the GC50 glass fiber filter was preliminarily measured by drying the filter in the thermostatic drier at 105° C. for one hour. The dry weight of the green algae was obtained by subtracting the dry weight of the filter that was preliminarily measured from the weight of the dried filter to which the green algae were adsorbed. Moreover, the amount of astaxanthin per dry weight was calculated from the measured value of the amount of astaxanthin in the given volume of the culture suspension.

### Example 1

### Preparation of Encysted Green Algae

Haematococcus pluvialis K0084 strains were inoculated into a medium (low nutrient medium) containing the components shown in Table 1 below.

TABLE 1

| Components | g/L |
|---|---|
| KNO₃ | 0.41 |
| K₂HPO₄ | 0.04 |
| MgSO₄•7H₂O | 0.075 |
| CaCl₂•2H₂O | 0.036 |
| Citric acid (anhydrous) | 0.006 |
| Ammonium iron (III) citrate | 0.006 |
| EDTA•2Na | 0.001 |
| Na₂CO₃ | 0.02 |
| CuSO₄•5H₂O | 0.00286 |
| H₃BO₄ | 0.00181 |
| MnCl₂•4H₂O | 0.00022 |
| ZnSO₄•7H₂O | 0.00008 |
| Na₂MoO₄ | 0.000021 |
| Co(NO₃)₂•6H₂O | 0.000000494 |

More specifically, 1 L of the low nutrient medium was placed in a 1.5 L flat culture flask, and the encysted K0084 strains were inoculated into the medium. The K0084 strains were cultivated for 7 days at 25° C. under irradiation with light at a photosynthetically active photon flux input of 25000 μmol-p/m³/s using a white fluorescent lamp, while bubbling a gas containing 3 vol % of CO₂ into the medium at a rate of 0.5 L/min (i.e., at an aeration rate of 0.5 vvm). After 7 days, the encysted K0084 strains were collected, and adjusted so that the concentration of the encysted K0084 strains in the low nutrient medium was 1.5×10⁶ cells/mL.

### (Main Culture)

First, 9 L of the low nutrient medium were placed in a flat culture tank in which acrylic transparent plates are opposite to each other so that the distance between the inner walls of the culture tank was 3 cm, and 1 L of the above-described encysted K0084 strains were inoculated into the medium so that the concentration was 1.5×10⁵ cells/mL to start cultivation. The encysted K0084 strains were cultivated for 21 days at 25° C. under irradiation with light at a photosynthetically active photon flux input of 25000 μmol-p/m³/s using six white fluorescent lamps provided on each side of the flat culture tank, while bubbling a gas containing 3 vol % of CO₂ into the medium at a rate of 5 L/min (i.e., at an aeration rate of 0.5 vvm). The amount of astaxanthin in the culture suspension, the number of cells, and the dry weight of algal cells collected from the culture suspension were measured regularly by the above-described methods to obtain the astaxanthin amount per cell. The results are shown in Table 2.

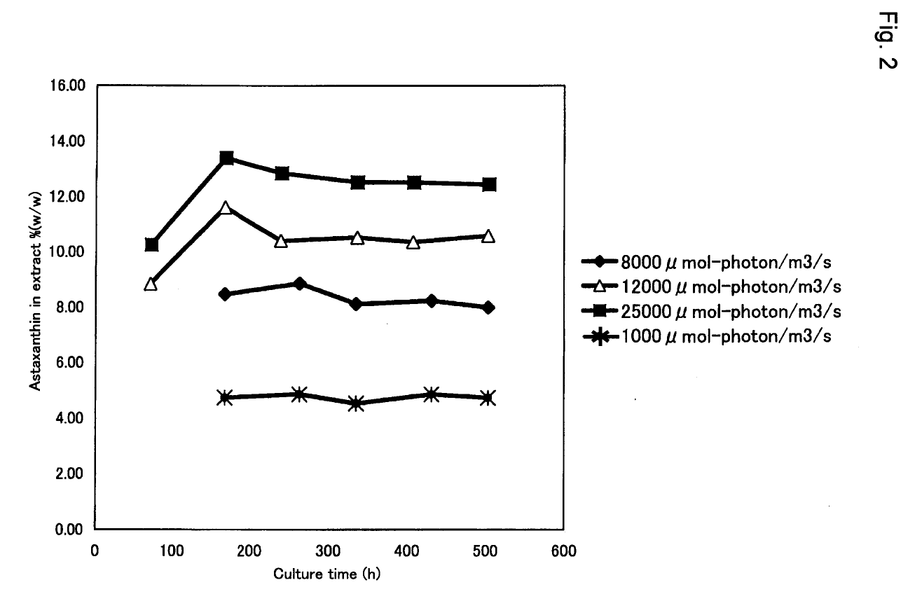

Next, 50 mL of the culture suspension was regularly collected, washed, and taken into a microtube designed for exclusive use with a bead beater. After adding zirconia beads to the tube, acetone was added thereto, and this was beaten with the bead beater. The beaten product was separated into a supernatant and a precipitate by centrifugation, and the supernatant was collected. Acetone was added again to the precipitate, and the same operation as described above was repeated until the color of the precipitate became almost completely white. The collected acetone fractions were combined to give a green algal extract. The astaxanthin concentration in the extract was measured as described above. The results are shown in Table 2 and FIG. 2.

### Example 2

The green algae was cultivated in the same manner as in Example 1, except that irradiation with light was performed at a photosynthetically active photon flux input of 12000 μmol-p/m³/s, to give a green algal extract, and the astaxanthin concentration in the extract was measured. The results are shown in Table 2 and FIG. 2.

### Example 3

The green algae was cultivated in the same manner as in Example 1, except that irradiation with light was performed at a photosynthetically active photon flux input of 8000 μmol-p/m³/s, to give a green algal extract, and the astaxanthin concentration in the extract was measured. The results are shown in Table 2 and FIG. 2.

### Comparative Example 1

The green algae was cultivated in the same manner as in Example 1, except that irradiation with light was performed at a photosynthetically active photon flux input of 1000 μmol-p/m³/s, to give a green algal extract, and the astaxanthin concentration in the extract was measured. The results are shown in Table 2 and FIG. 2.

TABLE 2

| | | Astaxanthin concentration on day 21 | | | | |
|---|---|---|---|---|---|---|
| | Photosynthetically | | % | % | | % |
| | active photon flux | | (w/w per wet | (w/w per dry | mg/L culture | (w/w per |
| | input*¹ | pg/cell | algal cells) | algal cells) | suspension | extract) |
| Example 1 | 25000 | 720 | 2.7 | 6.7 | 205 | 12.4 |
| Example 2 | 12000 | 650 | 1.8 | 4.7 | 140 | 10.6 |
| Example 3 | 8000 | 250 | 1.3 | 3.1 | 120 | 8.0 |
| Comparative | 1000 | 30 | 0.7 | 1.7 | 15 | 4.6 |
| Example 1 | | | | | | |

*¹.mol-p/m³/s

From Table 2 and FIG. 2, it can be found that by cultivating green algae under irradiation with light at a photosynthetically active photon flux input of 8000 μmol-p/m³/s or more, it is possible to obtain a green algal extract that contains astaxanthin at a concentration of 8 wt % or more. It is also found that a greater photosynthetically active photon flux input results in a higher concentration of astaxanthin in the extract. Further, it is also found that if the astaxanthin concentration of the dry algal cells is at least 3.1 wt %, then it is possible to obtain a green algal extract that contains astaxanthin at a concentration of 8 wt % or more.

### INDUSTRIAL APPLICABILITY

According to the present invention, a green algal extract containing astaxanthin at a higher concentration than conventional extracts is provided. Thus, the green algal extract of the present invention can be used in fields such as antioxidants, foods, pharmaceuticals, and cosmetics as is without further purification. Alternatively, by using the green algal extract of the present invention, it is possible to improve significantly the production efficiency of purified astaxanthin compared to ever before. Therefore, it is very useful in methods for the efficient production of astaxanthin.

1. A green algal extract, which contains astaxanthin at a concentration of 8 wt % or more.

2. The green algal extract of claim 1, wherein the green alga is a unicellular alga belonging to the genus Haematococcus.

3. The green algal extract of claim 1, wherein the green alga is Haematococcus pluvialis.

4. The green algal extract of claim 1, wherein the green alga is cultivated in an autotrophic medium.

5. A method for producing a green algal extract that contains astaxanthin at a concentration of 8 wt % or more, comprising:
cultivating an encysted green alga in a nutrient medium while supplying carbon dioxide and providing irradiation with light at a photosynthetically active photon flux input of 8000 μmol-photon/m³/s or more; and
extracting an oil component that contains astaxanthin.

6. The method of claim 5, wherein the nutrient medium is an autotrophic medium.

7. The method of claim 5, wherein the photosynthetically active photon flux input is not more than 240000 μmol-photon/m³/s.

8. The method of claim 5, wherein the green alga is a unicellular alga belonging to the genus Haematococcus.

9. The method of claim 5, wherein the green alga is Haematococcus pluvialis.

10. A method for producing astaxanthin, comprising:
cultivating an encysted green alga in a nutrient medium while supplying carbon dioxide and providing irradiation with light at a photosynthetically active photon flux input of 8000 μmol-photon/m³/s or more;
extracting an oil component that contains astaxanthin; and
recovering astaxanthin from the extracted oil component that contains astaxanthin.

11. The method of claim 10, wherein the nutrient medium is an autotrophic medium.

12. The method of claim 10, wherein the photosynthetically active photon flux input is not more than 240000 μmol-photon/m³/s.

13. The method of claim 10, wherein the green alga is a unicellular alga belonging to the genus Haematococcus.

14. The method of claim 10, wherein the green alga is Haematococcus pluvialis.

15. A green algal extract, which is obtained from a dried green alga that contains astaxanthin at a concentration of 3.1 wt % or more.

16. The green algal extract of claim 15, wherein astaxanthin is contained at a concentration of 8 wt % or more.

17. The green algal extract of claim 15, wherein the green alga is cultivated in an autotrophic medium.
